(19) United States
(12) Patent Application Publication
(10) Pub. No.: US 2026/0325298 A1
(43) Pub. Date: Oct. 22, 2026

(54) DELIVERY DEVICE FOR A REPLACEMENT HEART VALVE IMPLANT

(71) Applicant: BOSTON SCIENTIFIC SCIMED, INC., MAPLE GROVE, MN (US)

(72) Inventors: Kristi Mae Flury, Maple Grove, MN (US); Jessica D. Morlock, Hopkins, MN (US); Joseph Edward Adriaens, Elk River, MN (US)

(73) Assignee: BOSTON SCIENTIFIC SCIMED, INC., MAPLE GROVE, MN (US)

(21) Appl. No.: 19/651,003

(22) Filed: Apr. 17, 2026

Related U.S. Application Data
(60) Provisional application No. 63/790,342, filed on Apr. 17, 2025.

(51) Int. Cl. A61F 2/24

(52) U.S. Cl. CPC A61F 2/2436

(57) ABSTRACT

A delivery device for a replacement heart valve implant includes a shaft assembly including an implant holding portion configured to constrain the implant in a collapsed configuration. The shaft assembly includes a stent holder including a projection configured to engage a distal portion of a framework of the implant when the implant is constrained within the implant holding portion. The stent holder includes a visual contrast region proximate the projection. The projection may include a colored tip. The stent holder may include a plurality of projections configured to engage the framework of the implant when the implant is constrained within the implant holding portion and visual contrast regions at least partially surrounding each projection.

### CROSS-REFERENCE TO RELATED APPLICATIONS

This application claims the benefit of priority of U.S. Provisional Application No. 63/790,342 filed Apr. 17, 2025, the entire disclosure of which is hereby incorporated by reference.

### TECHNICAL FIELD

The disclosure relates generally to medical devices and more particularly to medical devices that are adapted for delivering and implanting stents and medical devices including a stent component, such as a replacement heart valve implant.

### BACKGROUND

A wide variety of intracorporeal medical devices and/or implants have been developed for medical use including artificial heart valve implants for repair or replacement of diseased heart valves. Due to the small size of the implants and delivery devices, and/or low light conditions during loading of the implant during a procedure (for example, because fluoroscopy is being performed), coupling of the implant to the delivery device may be difficult. Additionally, for those same reasons, verification of proper coupling may be difficult, which could lead to uncertainty during the procedure that the implant will deploy properly. Further still, some users may perform a low volume of cases, and familiarity with the delivery device may be limited. Confusion, cognitive load, and/or stress may slow and/or complicate the procedure. Of the known medical devices, systems, and methods, each has certain advantages and disadvantages. There is an ongoing need to provide alternative devices, systems, and methods to ensure proper loading of medical devices and/or heart valve implants into a delivery device.

### SUMMARY

In one example, a delivery device for a replacement heart valve implant may comprise an elongate shaft assembly including an implant holding portion. The implant holding portion may be configured to constrain a replacement heart valve implant in a radially collapsed configuration. The elongate shaft assembly may comprise a stent holder including a projection configured to engage a distal portion of an expandable framework of the replacement heart valve implant when the replacement heart valve implant is constrained within the implant holding portion of the implant delivery system. The stent holder may comprise a visual contrast region proximate the projection.

In addition, or alternatively, to any example disclosed herein, the visual contrast region is visible without electronic imaging equipment.

In addition, or alternatively, to any example disclosed herein, the visual contrast region is disposed on an outer surface of the stent holder.

In addition, or alternatively, to any example disclosed herein, the visual contrast region is etched onto the outer surface of the stent holder.

In addition, or alternatively, to any example disclosed herein, the visual contrast region at least partially surrounds the projection.

In addition, or alternatively, to any example disclosed herein, the visual contrast region extends more than halfway around the projection.

In addition, or alternatively, to any example disclosed herein, a contrast ratio of the visual contrast region to an outward facing surface of the projection is at least 3:1.

In addition, or alternatively, to any example disclosed herein, the contrast ratio of the visual contrast region to the outward facing surface of the projection is at least 7:1.

In addition, or alternatively, to any example disclosed herein, the contrast ratio of the visual contrast region to the outward facing surface of the projection is at least 12:1.

In addition, or alternatively, to any example disclosed herein, the visual contrast region has a darker visual appearance than the projection.

In addition, or alternatively, to any example disclosed herein, the stent holder is formed from a metallic material.

In addition, or alternatively, to any example disclosed herein, the metallic material comprises a pigment.

In addition, or alternatively, to any example disclosed herein, the visual contrast region comprises a pigment.

In addition, or alternatively, to any example disclosed herein, the visual contrast region comprises a matte finish.

In addition, or alternatively, to any example disclosed herein, and in a second example, a delivery device for a replacement heart valve implant may comprise an elongate shaft assembly including an implant holding portion. The implant holding portion may be configured to constrain a replacement heart valve implant in a radially collapsed configuration. The elongate shaft assembly may comprise a stent holder including a projection configured to engage a distal portion of an expandable framework of the replacement heart valve implant when the replacement heart valve implant is constrained within the implant holding portion of the implant delivery system. The projection may comprise a colored tip.

In addition, or alternatively, to any example disclosed herein, the colored tip comprises a paint disposed on the projection.

In addition, or alternatively, to any example disclosed herein, the colored tip is configured to be visible when the distal portion of the expandable framework of the replacement heart valve implant is engaged with the projection.

In addition, or alternatively, to any example disclosed herein, a delivery device for a replacement heart valve implant may comprise an elongate shaft assembly including an implant holding portion. The implant holding portion may be configured to constrain a replacement heart valve implant in a radially collapsed configuration. The elongate shaft assembly may comprise a stent holder including a plurality of projections configured to engage a distal portion of an expandable framework of the replacement heart valve implant when the replacement heart valve implant is constrained within the implant holding portion of the implant delivery system. The stent holder may comprise visual contrast regions at least partially surrounding each projection of the plurality of projections.

In addition, or alternatively, to any example disclosed herein, the visual contrast regions comprise etching on an outer surface of the stent holder.

In addition, or alternatively, to any example disclosed herein, a relative luminance of the plurality of projections divided by a relative luminance of the visual contrast regions at least partially surrounding each projection is at least 3.

The above summary of some embodiments, aspects, and/or examples is not intended to describe each disclosed embodiment or every implementation of the present disclosure. The figures and detailed description which follow more particularly exemplify these embodiments.

### BRIEF DESCRIPTION OF THE DRAWINGS

The disclosure may be more completely understood in consideration of the following detailed description in connection with the accompanying drawings, in which:

FIG. 1 schematically illustrates selected aspects of a replacement heart valve implant;

FIGS. 2-3 schematically illustrate selected aspects of a replacement heart valve system including the replacement heart valve implant and a delivery device;

FIG. 4 illustrates side and end views of a stent holder associated with the delivery device; and

FIGS. 5A-5B depict formulas for calculating relative luminance and contrast ratio.

While aspects of the disclosure are amenable to various modifications and alternative forms, specifics thereof have been shown by way of example in the drawings and will be described in detail. It should be understood, however, that the intention is not to limit aspects of the disclosure to the particular embodiments described. On the contrary, the intention is to cover all modifications, equivalents, and alternatives falling within the spirit and scope of the disclosure.

### DETAILED DESCRIPTION

The following description should be read with reference to the drawings, which are not necessarily to scale, wherein like reference numerals indicate like elements throughout the several views. The detailed description and drawings are intended to illustrate but not limit the disclosure. Those skilled in the art will recognize that the various elements described and/or shown may be arranged in various combinations and configurations without departing from the scope of the disclosure. The detailed description and drawings illustrate example embodiments of the disclosure.

For the following defined terms, these definitions shall be applied, unless a different definition is given in the claims or elsewhere in this specification.

All numeric values are herein assumed to be modified by the term “about,” whether or not explicitly indicated. The term “about”, in the context of numeric values, generally refers to a range of numbers that one of skill in the art would consider equivalent to the recited value (e.g., having the same function or result). In many instances, the term “about” may include numbers that are rounded to the nearest significant figure. Other uses of the term “about” (e.g., in a context other than numeric values) may be assumed to have their ordinary and customary definition(s), as understood from and consistent with the context of the specification, unless otherwise specified.

The recitation of numerical ranges by endpoints includes all numbers within that range, including the endpoints (e.g., 1 to 5 includes 1, 1.5, 2, 2.75, 3, 3.80, 4, and 5).

Although some suitable dimensions, ranges, and/or values pertaining to various components, features and/or specifications are disclosed, one of skill in the art, incited by the present disclosure, would understand desired dimensions, ranges, and/or values may deviate from those expressly disclosed.

As used in this specification and the appended claims, the singular forms “a”, “an”, and “the” include plural referents unless the content clearly dictates otherwise. As used in this specification and the appended claims, the term “or” is generally employed in its sense including “and/or” unless the content clearly dictates otherwise. It is to be noted that to facilitate understanding, certain features of the disclosure may be described in the singular, even though those features may be plural or recurring within the disclosed embodiment(s). Each instance of the features may include and/or be encompassed by the singular disclosure(s), unless expressly stated to the contrary. For example, a reference to one feature may be equally referred to all instances and quantities beyond one of said feature unless clearly stated to the contrary. As such, it will be understood that the following discussion may apply equally to any and/or all components for which there are more than one within the device, etc. unless explicitly stated to the contrary.

Relative terms such as “proximal”, “distal”, “advance”, “retract”, variants thereof, and the like, may be generally considered with respect to the positioning, direction, and/or operation of various elements relative to a user/operator/manipulator of the device, wherein “proximal” and “retract” indicate or refer to closer to or toward the user and “distal” and “advance” indicate or refer to farther from or away from the user. In some instances, the terms “proximal” and “distal” may be arbitrarily assigned to facilitate understanding of the disclosure, and such instances will be readily apparent to the skilled artisan. Other relative terms, such as “upstream”, “downstream”, “inflow”, and “outflow” refer to a direction of fluid flow within a lumen, such as a body lumen, a blood vessel, or within a device. Still other relative terms, such as “axial”, “circumferential”, “longitudinal”, “lateral”, “radial”, etc. and/or variants thereof generally refer to direction and/or orientation relative to a central longitudinal axis of the disclosed structure or device.

The term “extent” may be understood to mean the greatest measurement of a stated or identified dimension, unless the extent or dimension in question is preceded by or identified as a “minimum”, which may be understood to mean the smallest measurement of the stated or identified dimension. For example, “outer extent” may be understood to mean an outer dimension, “radial extent” may be understood to mean a radial dimension, “longitudinal extent” may be understood to mean a longitudinal dimension, etc. Each instance of an “extent” may be different (e.g., axial, longitudinal, lateral, radial, circumferential, etc.) and will be apparent to the skilled person from the context of the individual usage. Generally, an “extent” may be considered a greatest possible dimension measured according to the intended usage, while a “minimum extent” may be considered a smallest possible dimension measured according to the intended usage. In some instances, an “extent” may generally be measured orthogonally within a plane and/or cross-section, but may be, as will be apparent from the particular context, measured differently – such as, but not limited to, angularly, radially, circumferentially (e.g., along an arc), etc.

The terms “monolithic” and “unitary” shall generally refer to an element or elements made from or consisting of a single structure or base unit/element. A monolithic and/or unitary element shall exclude structure and/or features made by assembling or otherwise joining multiple discrete structures or elements together.

It is noted that references in the specification to “an embodiment”, “some embodiments”, “other embodiments”, etc., indicate that the embodiment(s) described may include a particular feature, structure, or characteristic, but every embodiment may not necessarily include the particular feature, structure, or characteristic. Moreover, such phrases are not necessarily referring to the same embodiment. Further, when a particular feature, structure, or characteristic is described in connection with an embodiment, it would be within the knowledge of one skilled in the art to implement the particular feature, structure, or characteristic in connection with other embodiments, whether or not explicitly described, unless clearly stated to the contrary. That is, the various individual elements described below, even if not explicitly shown in a particular combination, are nevertheless contemplated as being combinable or arrangeable with each other to form other additional embodiments or to complement and/or enrich the described embodiment(s), as would be understood by one of ordinary skill in the art.

For the purpose of clarity, certain identifying numerical nomenclature (e.g., first, second, third, fourth, etc.) may be used throughout the description and/or claims to name and/or differentiate between various described and/or claimed features. It is to be understood that the numerical nomenclature is not intended to be limiting and is exemplary only. In some embodiments, alterations of and deviations from previously used numerical nomenclature may be made in the interest of brevity and clarity. That is, a feature identified as a “first” element may later be referred to as a “second” element, a “third” element, etc. or may be omitted entirely, and/or a different feature may be referred to as the “first” element. The meaning and/or designation in each instance will be apparent to the skilled practitioner.

Additionally, it should be noted that in any given figure, some features may not be shown, or may be shown schematically, for clarity and/or simplicity. Additional details regarding some components and/or method steps may be illustrated in other figures in greater detail. The devices and/or methods disclosed herein may provide a number of desirable features and benefits as described in more detail below.

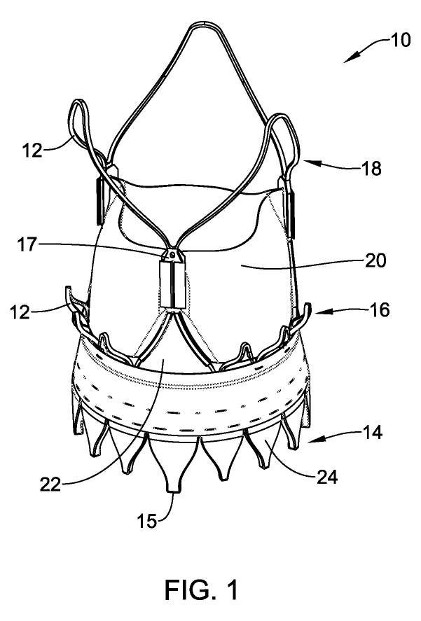

FIG. 1 illustrates selected aspects of a replacement heart valve implant 10. It should be appreciated that the replacement heart valve implant 10 can be any type of replacement heart valve (e.g., a mitral valve, an aortic valve, etc.). Some non-limiting examples of the replacement heart valve implant 10 may include the ACURATE NEO2™, the ACURATE PRIME™, and/or family members thereof from Boston Scientific. Other examples are also contemplated. In use, the replacement heart valve implant 10 may be implanted (e.g., surgically or through transcatheter delivery) within a native heart valve in a mammalian heart. The replacement heart valve implant 10 can be configured to allow one-way flow through the replacement heart valve implant 10 from an inflow end to an outflow end.

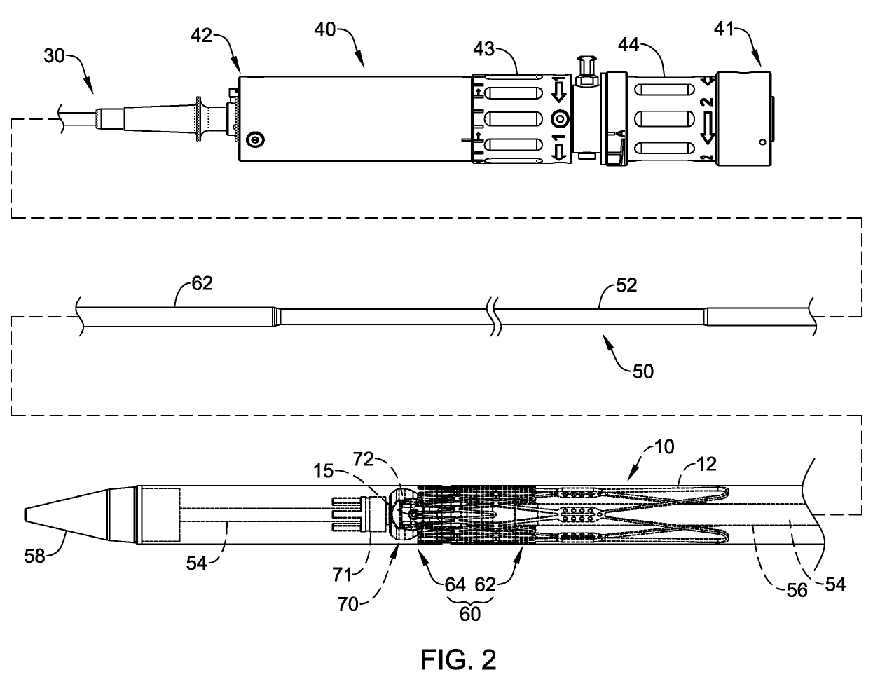

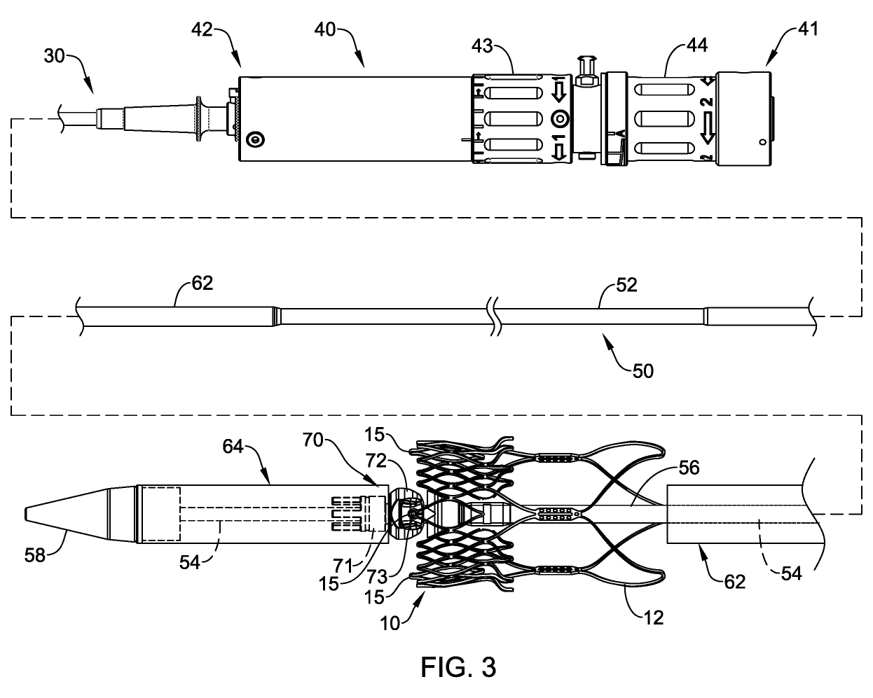

The replacement heart valve implant 10 may include an expandable framework 12 defining a central lumen. In some embodiments, the expandable framework 12 may have a substantially circular cross-section. In some embodiments, the expandable framework 12 can have a non-circular (e.g., D-shaped, elliptical, etc.) cross-section. Some suitable but non-limiting examples of materials that may be used to form the expandable framework 12, including but not limited to metals and metal alloys, composites, ceramics, polymers, and the like, are described below. The replacement heart valve implant 10 and/or the expandable framework 12 may be configured to shift between a radially collapsed configuration (e.g., FIG. 2) and a radially expanded configuration (e.g., FIG. 3). In some embodiments, the expandable framework 12 may be self-expanding. In some embodiments, the expandable framework 12 may be self-biased toward the radially expanded configuration. In some embodiments, the expandable framework 12 may be mechanically expandable. In some embodiments, the expandable framework 12 may be balloon expandable. Other configurations, including combinations thereof, are also contemplated.

In some embodiments, the expandable framework 12 may define a lower crown 14 proximate and/or at an inflow end, an upper crown 16 proximate and/or at an outflow end, and a plurality of stabilization arches 18 extending downstream from the outflow end. The expandable framework may comprise a body portion extending between the lower crown 14 and the upper crown 16. In at least some embodiments, the body portion may be formed as and/or similar to a stent or a stent-like structure. In some embodiments, the body portion may comprise a plurality of closed cells. In some embodiments, the expandable framework 12 may comprise a plurality of commissure posts 17. In some embodiments, the plurality of commissure posts 17 may extend downstream of and/or away from the upper crown 16. In some embodiments, the plurality of stabilization arches 18 may extend downstream of and/or away from the upper crown 16 and/or the plurality of commissure posts 17 in a direction opposite the lower crown 14. In some embodiments, the upper crown 16 and/or the plurality of commissure posts 17 may be disposed longitudinally and/or axially between the lower crown 14 and the plurality of stabilization arches 18. In some embodiments, the lower crown 14 may comprise a hook 15 and/or a plurality of hooks (e.g., ref. 15), as described herein.

In some embodiments, the replacement heart valve implant 10 and/or the expandable framework 12 may include a proximal portion and a distal portion. In some embodiments, orientation of the replacement heart valve implant 10 may be related to a delivery device 30 (e.g., FIGS. 2-3) and/or a direction of implantation relative to a treatment site (e.g., a native heart valve). In some embodiments, the proximal portion may include the outflow end and/or the plurality of stabilization arches 18. In some embodiments, the proximal portion may include the upper crown 16 and/or the plurality of commissure posts 17. In some embodiments, the distal portion may include the inflow end, the lower crown 14, and/or the hook 15. In some embodiments, the distal portion may include the body portion and/or a portion of the body portion disposed immediately adjacent the lower crown 14. Other configurations are also contemplated.

In some embodiments, the replacement heart valve implant 10 may include a plurality of valve leaflets 20 disposed within the central lumen. The plurality of valve leaflets 20 may be coupled, secured, and/or fixedly attached to the expandable framework 12 at the plurality of commissure posts 17 to form and/or define a plurality of commissures. In addition, or alternatively, in some embodiments, the plurality of valve leaflets 20 may be coupled, secured, and/or fixedly attached to the expandable framework 12 proximate and/or at other locations, such as the inflow end, the lower crown 14, etc. The plurality of valve leaflets 20 may be configured to shift between an open position and a closed position. The plurality of valve leaflets 20 may be configured to substantially restrict fluid flow through the replacement heart valve implant 10 in the closed position. The plurality of valve leaflets 20 may move apart from each other and/or radially outward within the central lumen in the open position to permit fluid flow through the replacement heart valve implant 10 and/or the central lumen.

In some embodiments, the plurality of valve leaflets 20 may be comprised of a polymer, such as a thermoplastic polymer. In some embodiments, the plurality of valve leaflets 20 may include at least 50 percent by weight of a polymer. In some embodiments, the plurality of valve leaflets 20 may be formed from porcine pericardium, bovine pericardium, or other tissue. Other configurations and/or materials are also contemplated.

In some embodiments, the replacement heart valve implant 10 may include an inner skirt 22 disposed on and/or extending along an inner surface of the expandable framework 12. In at least some embodiments, the inner skirt 22 may be fixedly attached to the expandable framework 12. The inner skirt 22 may direct fluid, such as blood, flowing through the replacement heart valve implant 10 toward the plurality of valve leaflets 20. In at least some embodiments, the inner skirt 22 may be fixedly attached to and/or integrally formed with the plurality of valve leaflets 20. The inner skirt 22 may ensure the fluid flows through the central lumen of the replacement heart valve implant 10 and does not flow around the plurality of valve leaflets 20 when they are in the closed position.

In some embodiments, the replacement heart valve implant 10 may include an outer skirt 24 disposed on and/or extending along an outer surface of the expandable framework 12. In some embodiments, the outer skirt 24 may be disposed at and/or adjacent the lower crown 14. The outer skirt 24 may ensure the fluid flows through the replacement heart valve implant 10 and does not flow around the replacement heart valve implant 10 (e.g., between the expandable framework 12 and the vessel wall).

In some embodiments, the inner skirt 22 and/or the outer skirt 24 may include a polymer, and/or may include at least 50 percent by weight of a polymer. In some embodiments, the inner skirt 22 and/or the outer skirt 24 may be substantially impervious to fluid. In some embodiments, the inner skirt 22 and/or the outer skirt 24 may be formed from a thin tissue (e.g., porcine pericardium, bovine pericardium, or other tissue, etc.), a coated fabric material, or a nonporous and/or impermeable fabric material. Other configurations are also contemplated. Some suitable but non-limiting examples of materials that may be used to form the inner skirt 22 and/or the outer skirt 24 including but not limited to polymers, composites, and the like, are described below.

In some embodiments, the inner skirt 22 and/or the outer skirt 24 may seal one of, some of, a plurality of, or each of a plurality of interstices formed in the expandable framework 12. In at least some embodiments, sealing the interstices may be considered to prevent fluid from flowing through the interstices of the expandable framework 12. In some embodiments, the inner skirt 22 and/or the outer skirt 24 may be attached to the expandable framework 12 using one or more methods including but not limited to tying with sutures or filaments, adhesive bonding, melt bonding, embedding or over molding, welding, etc.

In some embodiments, the expandable framework 12 and/or the replacement heart valve implant 10 may have an outer extent of about 17 millimeters (mm) (0.669 inches), about 19.5 mm (0.768 inches), about 23 mm (0.905 inches), about 25 mm (0.984 inches), about 27 mm (1.063 inches), about 30 mm (1.181 inches), about 31 mm (1.220 inches), etc. in an unconstrained configuration (e.g., in the radially expanded configuration). In some embodiments, the expandable framework 12 and/or the replacement heart valve implant 10 may have an outer extent of about 11 mm (0.433 inches), about 10 mm (0.394 inches), about 9 mm (0.354 inches), about 8 mm (0.315 inches), about 7 mm (0.276 inches), about 6 mm (0.236 inches), about 5 mm (0.197 inches), about 4 mm (0.157 inches), etc. in the radially collapsed configuration. Other configurations are also contemplated.

FIGS. 2-3 illustrate selected aspects of a replacement heart valve system comprising the replacement heart valve implant 10 and a delivery device 30 for delivering the replacement heart valve implant 10 to a treatment site (e.g., native heart valve). It should be noted that FIGS. 2-3 may include at least one change of scale (e.g., all parts of the figure are not drawn to the same scale) to improve viewability and show additional detail of selected aspects of the delivery device 30. Additionally, the expandable framework 12 is shown and some elements of the replacement heart valve implant 10 are omitted from FIGS. 2-3 to improve clarity. It should further be noted that all elements are not shown or labelled in each figure in the interest of clarity.

The delivery device 30 may include a handle assembly 40 and an elongate shaft assembly 50 extending distally from the handle assembly 40. The handle assembly 40 may include a proximal end 41 and a distal end 42 opposite the proximal end 41. The elongate shaft assembly 50 may extend distally from the distal end 42 of the handle assembly 40. In some embodiments, the handle assembly 40 may include one or more rotatable knobs. In some embodiments, the one or more rotatable knobs may include a first rotatable knob 43 and a second rotatable knob 44. In at least some embodiments, the first rotatable knob 43 and/or the second rotatable knob 44 may be configured to rotate around a central longitudinal axis of the delivery device 30 and/or the handle assembly 40. Other configurations and/or types of interface controls are also contemplated.

In some embodiments, rotation of the first rotatable knob 43 around the central longitudinal axis of the handle assembly 40 may be configured to deploy the proximal portion of the replacement heart valve implant 10 from an implant holding portion 60 of the delivery device 30. Similarly, rotation of the second rotatable knob 44 around the central longitudinal axis of the handle assembly 40 may be configured to deploy the distal portion of the replacement heart valve implant 10 from the implant holding portion 60 of the delivery device 30.

In some embodiments, a distal portion of the delivery device 30 and/or the elongate shaft assembly 50 may comprise the implant holding portion 60. The implant holding portion 60 may be configured to engage with and/or constrain the replacement heart valve implant 10 and/or the expandable framework 12 in the radially collapsed configuration. The elongate shaft assembly 50 may include an outer tubular member 52 secured to and extending distally from the handle assembly 40, and an inner shaft 54 secured to and extending distally from the handle assembly 40 within the outer tubular member 52 to a distal tip 58 disposed distal of the implant holding portion 60. In some embodiments, the implant holding portion 60 may comprise a proximal sheath 62 and a distal sheath 64. In some embodiments, in a delivery configuration of the implant holding portion 60 (e.g., FIG. 2), the implant holding portion 60 may be configured to constrain the replacement heart valve implant 10 and/or the expandable framework 12 within the proximal sheath 62 and the distal sheath 64. In some embodiments, the proximal sheath 62 and/or the distal sheath 64 may be formed from a polymeric material. In some embodiments, the proximal sheath 62 and/or the distal sheath 64 may include a reinforcing element (not shown) disposed therein and/or thereon. In some embodiments, the reinforcing element(s) may be embedded within the proximal sheath 62 and/or the distal sheath 64. In some embodiments, the reinforcing element(s) may be a coil, a mesh, one or more filaments, bands, or strips, or another suitable structure. Other configurations are also contemplated. In some embodiments, the reinforcing element(s) may be configured to prevent radial expansion of the proximal sheath 62 and/or the distal sheath 64.

In some embodiments, the inner shaft 54 may be slidably disposed within a lumen of the outer tubular member 52. In some embodiments, the elongate shaft assembly 50 may include an intermediate tubular member 56 disposed within and/or radially inward of the outer tubular member 52 and about, around, surrounding, and/or radially outward of the inner shaft 54. In at least some embodiments, the inner shaft 54 and the outer tubular member 52 are each axially translatable relative to the intermediate tubular member 56 independently of each other. For example, the inner shaft 54 may be translated relative to the intermediate tubular member 56 without translating the outer tubular member 52 relative to the intermediate tubular member 56, and vice versa.

In some embodiments, the proximal sheath 62 may be fixedly attached to the outer tubular member 52. In some embodiments, the proximal sheath 62 may be fixedly attached to and/or may extend distally from a distal end of the outer tubular member 52. In some embodiments, the proximal sheath 62 may have a constant inner diameter.

In some embodiments, the distal sheath 64 and/or the distal tip 58 may be fixedly attached to the inner shaft 54. In some embodiments, the distal sheath 64 may be fixedly attached to the distal tip 58. In some embodiments, the distal sheath 64 may extend proximally from the distal tip 58. In some embodiments, the distal sheath 64 may have a constant inner diameter. In some embodiments, the inner shaft 54 may include and/or at least partially define a guidewire lumen extending therethrough. In some embodiments, the guidewire lumen may extend through the handle assembly 40.

In some embodiments, the handle assembly 40 may be configured to manipulate and/or translate the proximal sheath 62 and/or the distal sheath 64 relative to each other using the first rotatable knob 43 and/or the second rotatable knob 44, as discussed further below, to shift the implant holding portion 60, and/or the proximal sheath 62 and the distal sheath 64, between a delivery configuration (e.g., FIG. 2) and a deployment configuration (e.g., FIG. 3). In some embodiments, the handle assembly 40 and/or the second rotatable knob 44 may be configured to manipulate and/or translate the inner shaft 54 and/or the distal sheath 64 relative to the elongate shaft assembly 50, the outer tubular member 52, the intermediate tubular member 56, and/or the proximal sheath 62. In some embodiments, the handle assembly 40 and/or the first rotatable knob 43 may be configured to manipulate and/or translate the outer tubular member 52 and/or the proximal sheath 62 relative to the elongate shaft assembly 50, the inner shaft 54, the intermediate tubular member 56, and/or the distal sheath 64.

During delivery of the replacement heart valve implant 10 to a treatment site (e.g., the native heart valve, the aortic valve, etc.), the replacement heart valve implant 10 may be disposed at least partially within the proximal sheath 62 and/or the distal sheath 64 in the radially collapsed configuration in the delivery configuration of the implant holding portion 60. In some embodiments, the proximal sheath 62 and/or the distal sheath 64 may collectively define the implant holding portion 60 of the delivery device 30. In some embodiments, the implant holding portion 60 may be configured to constrain the replacement heart valve implant 10 in the radially collapsed configuration when the implant holding portion 60 is in the delivery configuration.

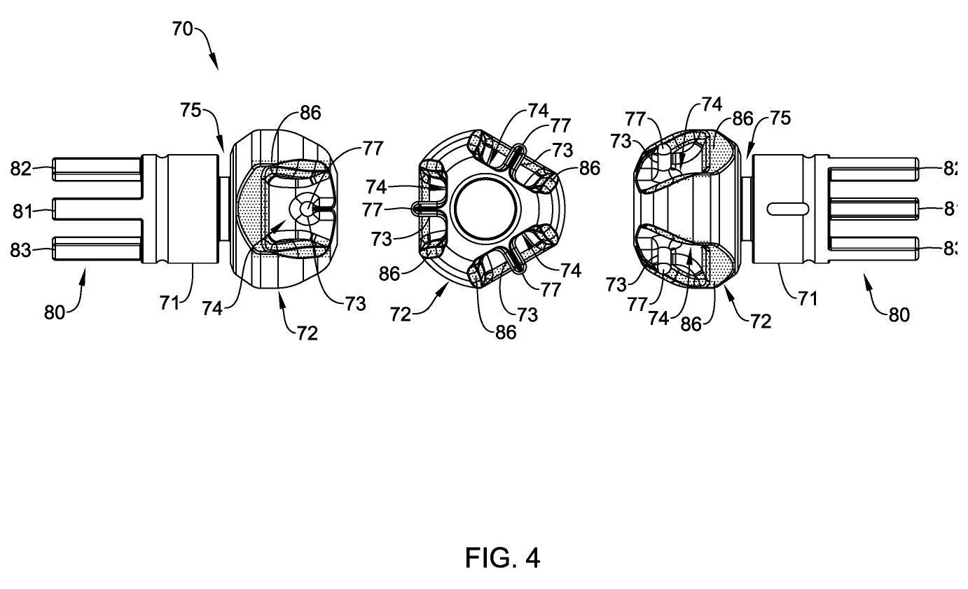

In some embodiments, the implant holding portion 60 of the delivery device 30 may comprise a stent holder 70. Selected aspects of the stent holder 70 are shown in more detail in FIG. 4, which illustrates side and end views of a stent holder associated with the delivery device. In some embodiments, the replacement heart valve implant 10 may be releasably coupled to and/or releasably engaged with the intermediate tubular member 56 and/or the stent holder 70 when the replacement heart valve implant 10 is constrained within the implant holding portion 60 of the delivery device 30 in the radially collapsed configuration (e.g., FIG. 2).

In some embodiments, the stent holder 70 may comprise a proximal head 72 having a first outermost radial extent and a distal tail 71 having a second outermost radial extent less than the first outermost radial extent. In some embodiments, the proximal head 72 may have and/or may comprise a generally bulbous shape. In at least some embodiments, the second outermost radial extent may be substantially constant along an entire length of the distal tail 71. Other configurations are also contemplated.

In some embodiments, the stent holder 70 may be fixedly attached to the elongate shaft assembly 50 (e.g., FIGS. 2-3). In some embodiments, the stent holder 70 may be fixedly attached to the intermediate tubular member 56 of the elongate shaft assembly 50. In some embodiments, the stent holder 70 may include a lumen extending longitudinally and/or axially therethrough. In at least some embodiments, at least a portion of the elongate shaft assembly 50 may extend longitudinally and/or axially through the lumen of the stent holder 70.

In some embodiments, the stent holder 70 may be configured and/or adapted to be visible under fluoroscopy. In at least some embodiments, the stent holder 70 may be formed from a metallic material. In one non-limiting example, the stent holder 70 may be formed from stainless steel. In some alternative embodiments, the stent holder 70 may be formed from a polymeric material, a composite material, etc. Some non-limiting examples of suitable materials may include polyetheretherketone (PEEK), polyimide (PI), nylon, glass-filled composite materials, etc. Some suitable but non-limiting materials for the stent holder 70 and/or components or elements thereof are described below. In some alternative embodiments, the stent holder 70 may be monolithically formed with the elongate shaft assembly 50 and/or the intermediate tubular member 56.

The stent holder 70 and/or the proximal head 72 may comprise a projection 73 configured to engage the expandable framework 12 and/or the lower crown 14 thereof in the radially collapsed configuration and/or when the replacement heart valve implant 10 is constrained within the implant holding portion 60 of the delivery device 30. In some embodiments, the stent holder 70 and/or the proximal head 72 may comprise a plurality of projections (e.g., ref. 73). The stent holder 70 and/or the proximal head 72 may comprise a recess 74 associated with the projection 73. In embodiments comprising a plurality of projections (e.g., ref. 73), the stent holder 70 and/or the proximal head 72 may comprise a plurality of recesses (e.g., ref. 74) associated with the plurality of projections (e.g., ref. 73). As such, in at least some embodiments, each projection may be associated with its own recess. In some embodiments, the projection 73 may extend radially outward from the proximal head 72 of the stent holder 70 and/or its associated recess (e.g., ref. 74). Similarly, the plurality of projections (ref. 73) may extend radially outward from the proximal head 72 of the stent holder 70 and/or their associated recesses.

When engaging and/or coupling the expandable framework 12 and/or the replacement heart valve implant 10 with the stent holder 70, a portion of the expandable framework 12 and/or the lower crown 14 may be disposed within the recess 74 with the projection 73 associated therewith extending through the expandable framework 12 and/or the lower crown 14 (e.g., the expandable framework 12 and/or the lower crown 14 may extend around the projection 73). In some embodiments, the portion of the expandable framework 12 and/or the lower crown 14 that may be disposed within the recess 74 and/or that may be engaged with the projection 73 may be a hook 15 (e.g., FIGS. 1-3). In some embodiments, the hook 15 may extend axially from the lower crown 14. In some embodiments, the hook 15 may be configured to extend around the projection 73 in the radially collapsed configuration. Similarly, when engaging and/or coupling the expandable framework 12 and/or the replacement heart valve implant 10 with the stent holder 70, several portions of the expandable framework 12 and/or the lower crown 14 may be disposed within the plurality of recesses (e.g., ref. 74) with the plurality of projections (e.g., ref. 73) associated therewith extending through the expandable framework 12 and/or the lower crown 14 (e.g., the expandable framework 12 and/or the lower crown 14 may extend around each projection of the plurality of projections (e.g., ref. 73)). In some embodiments, the several portions of the expandable framework 12 and/or the lower crown 14 that may be disposed within the plurality of recesses (e.g., ref. 74) and/or that may be engaged with the plurality of projections (e.g., ref. 73) may be a plurality of hooks (e.g., ref. 15). In some embodiments, the plurality of hooks (e.g., ref. 15) may extend axially from the lower crown 14. In some embodiments, the plurality of hooks (e.g., ref. 15) may be configured to extend around the plurality of projections (e.g., ref. 73) in the radially collapsed configuration.

In some embodiments, the stent holder 70 may comprise an annular recess 75 formed therein distal of the proximal head 72. In some embodiments, the annular recess 75 may be disposed immediately adjacent to the proximal head 72. In some embodiments, the annular recess 75 may be axially spaced apart from the proximal head 72. In some embodiments, the annular recess 75 may be configured and/or adapted to be visible to an imaging device as a void under fluoroscopy. In some embodiments, the annular recess 75 may have a width (e.g., a dimension in an axial direction of the delivery device 30) of about 0.3 millimeters to about 0.4 millimeters. In some embodiments, the annular recess 75 may have a radial depth defining a remaining radial material thickness of 0.07 +/- 0.01 millimeters. As such, after forming the annular recess 75, the remaining radial material thickness between the lumen of the stent holder 70 and a radially outward facing surface of the annular recess 75 is 0.07 +/- 0.01 millimeters. Other configurations and/or dimensions are also contemplated. In some embodiments, the remaining radial material thickness may be sized, configured, and/or adapted to perform mechanically while being thin enough that the annular recess 75 appears as a void under fluoroscopy when the imaging device is properly aligned with a reference plane associated with the native valve annulus. The annular recess 75 may be useful for identifying and/or avoiding viewpoint/observer relative misalignment of the replacement heart valve implant 10 with the native valve annulus (e.g., the parallax effect) during delivery and/or deployment.

In some embodiments, the stent holder 70 may comprise a plurality of longitudinal marker elements 80 configured to be visible under fluoroscopy with an imaging device. In some embodiments, the plurality of longitudinal marker elements 80 may include two longitudinal marker elements, three longitudinal marker elements, etc. In some embodiments, the plurality of longitudinal marker elements 80 may extend distally from the distal tail 71 of the stent holder 70. In some embodiments, the plurality of longitudinal marker elements 80 may extend distally from a distal end of the distal tail 71.

In some embodiments, the plurality of longitudinal marker elements 80 may be oriented parallel to a central longitudinal axis of the elongate shaft assembly 50. In some embodiments, the plurality of longitudinal marker elements 80 may be spaced radially outward from the inner shaft 54 of the elongate shaft assembly 50. In some embodiments, the plurality of longitudinal marker elements 80 may be equally spaced apart from each other circumferentially about the central longitudinal axis of the elongate shaft assembly 50.

In one example configuration, the plurality of longitudinal marker elements 80 may include a first longitudinal marker element 81, a second longitudinal marker element 82, and a third longitudinal marker element 83. In some embodiments, the first longitudinal marker element 81, the second longitudinal marker element 82, and the third longitudinal marker element 83 may be oriented parallel to a central longitudinal axis of the elongate shaft assembly 50. In some embodiments, the first longitudinal marker element 81, the second longitudinal marker element 82, and the third longitudinal marker element 83 may be spaced radially outward from the inner shaft 54 of the elongate shaft assembly 50. In some embodiments, the first longitudinal marker element 81, the second longitudinal marker element 82, and the third longitudinal marker element 83 may be equally spaced apart from each other circumferentially about the central longitudinal axis of the elongate shaft assembly 50. For example, the first longitudinal marker element 81, the second longitudinal marker element 82, and the third longitudinal marker element 83 may be arranged at 120 degrees apart from each other around the central longitudinal axis, and/or the first longitudinal marker element 81, the second longitudinal marker element 82, and the third longitudinal marker element 83 may be positioned and/or disposed on a theoretical circle around the central longitudinal axis and circumferentially spaced apart by an arc length equal to one third of a circumference of the theoretical circle.

In some embodiments, the plurality of longitudinal marker elements 80 may comprise a plurality of fingers extending distally from the distal tail 71 of the stent holder 70. In some embodiments, the plurality of fingers may include two fingers, three fingers, four fingers, etc. In one example, the plurality of fingers may include exactly three fingers.

In some embodiments, the plurality of longitudinal marker elements 80 and/or the plurality of fingers may extend away from the distal tail 71 of the stent holder 70. In some embodiments, the plurality of longitudinal marker elements 80 and/or the plurality of fingers may be longitudinally and/or axially aligned with the plurality of projections (e.g., ref. 73). In some embodiments, the plurality of longitudinal marker elements 80 and/or the plurality of fingers may be configured and/or adapted to align with the plurality of commissure posts 17 and/or the commissures of the replacement heart valve implant 10. In some embodiments, the plurality of longitudinal marker elements 80 and/or the plurality of fingers may be configured and/or adapted to be visible under fluoroscopy. In some embodiments, the plurality of longitudinal marker elements 80 and/or the plurality of fingers may be configured and/or adapted to align the delivery device 30, the stent holder 70, and/or the plurality of commissure posts 17 and/or the commissures of the replacement heart valve implant 10 with the native valve commissures of the native heart valve (e.g., the aortic valve) under fluoroscopy. In some embodiments, the plurality of longitudinal marker elements 80 and/or the plurality of fingers may be configured and/or adapted to rotationally align the delivery device 30, the stent holder 70, and/or the plurality of commissure posts 17 and/or the commissures of the replacement heart valve implant 10 with the native valve commissures of the native heart valve (e.g., the aortic valve) under fluoroscopy.

As discussed herein, in some embodiments, the stent holder 70 and/or the proximal head 72 thereof may be configured to engage the expandable framework 12 in the radially collapsed configuration and/or when the replacement heart valve implant 10 is constrained within the implant holding portion 60 of the delivery device 30. In some embodiments, in the delivery configuration of the implant holding portion 60, the distal sheath 64 may be configured to constrain at least a portion of the replacement heart valve implant 10 (e.g., at least a portion of the expandable framework 12 and/or the lower crown 14) in engagement with the proximal head 72, the projection 73, and/or the plurality of projections (e.g., ref. 73).

In some embodiments, the projection 73 and/or the plurality of projections (e.g., ref. 73) may be configured and/or adapted to engage the distal portion and/or the lower crown 14 of the expandable framework 12 of the replacement heart valve implant 10 in the radially collapsed configuration and/or when the replacement heart valve implant 10 is constrained within the implant holding portion 60 of the delivery device 30. In some embodiments, the projection 73 and/or the plurality of projections (e.g., ref. 73) may be configured to extend radially outward through the expandable framework 12 of the replacement heart valve implant 10 in the radially collapsed configuration and/or when the replacement heart valve implant 10 is constrained within the implant holding portion 60 of the delivery device 30.

Turning back to FIG. 2, in some embodiments, the proximal sheath 62 may be configured to cover the proximal portion of the replacement heart valve implant 10 in the radially collapsed configuration when the implant holding portion 60 is in the delivery configuration, and the distal sheath 64 may be configured to cover the distal portion of the replacement heart valve implant 10 in the radially collapsed configuration when the implant holding portion 60 is in the delivery configuration. In some embodiments, the replacement heart valve implant 10 may be constrained in the radially collapsed configuration by the proximal sheath 62 and the distal sheath 64 in the delivery configuration of the implant holding portion 60. In some embodiments, in the delivery configuration of the implant holding portion 60, the proximal head 72 of the stent holder 70 may be disposed within the distal sheath 64.

In some embodiments, the proximal sheath 62 may be disposed adjacent to the distal sheath 64 in the delivery configuration. In some embodiments, the proximal sheath 62 may abut the distal sheath 64 in the delivery configuration. In some embodiments, the proximal sheath 62 may be axially offset from the distal sheath 64 in the delivery configuration. In some embodiments, the proximal sheath 62 may be axially non-overlapping with the distal sheath 64 in the delivery configuration. In some embodiments, the proximal sheath 62 may be axially spaced apart from the distal sheath 64 in the delivery configuration.

In some embodiments, the proximal sheath 62 may be axially spaced apart from the distal sheath 64 in the delivery configuration by less than 20% of an overall length of the replacement heart valve implant 10 in the radially collapsed configuration. In some embodiments, the proximal sheath 62 may be axially spaced apart from the distal sheath 64 in the delivery configuration by less than 15% of an overall length of the replacement heart valve implant 10 in the radially collapsed configuration. In some embodiments, the proximal sheath 62 may be axially spaced apart from the distal sheath 64 in the delivery configuration by less than 10% of an overall length of the replacement heart valve implant 10 in the radially collapsed configuration. In some embodiments, the proximal sheath 62 may be axially spaced apart from the distal sheath 64 in the delivery configuration by less than 5% of an overall length of the replacement heart valve implant 10 in the radially collapsed configuration. Other configurations are also contemplated.

In at least some embodiments, the proximal sheath 62 and/or the distal sheath 64 may be formed from a material that is at least partially transparent (e.g., clear, “see-through”). In some embodiments, the proximal sheath 62 and/or the distal sheath 64 may be formed from a material that is completely transparent. In some embodiments, any reinforcing element disposed on, disposed within, and/or embedded within the proximal sheath 62 and/or the distal sheath 64 may be discontinuous and/or may be formed such that at least a portion of the proximal sheath 62 and/or the distal sheath 64 remains at least partially or completely transparent (e.g., clear, “see-through”). Other configurations are also contemplated.

It may be desirable and/or useful to be able to see the replacement heart valve implant 10 within the implant holding portion 60 when the replacement heart valve implant 10 is constrained therein in the delivery configuration. In particular, it may be desirable and/or useful to see the expandable framework 12 properly engaged with the projection 73 and/or the plurality of projections (e.g., ref. 73) of the stent holder 70 when the replacement heart valve implant 10 is constrained within the implant holding portion 60 in the delivery configuration. In some cases, the process of loading the replacement heart valve implant 10 into the delivery device 30 may take place in relatively low-light conditions, thereby making it more difficult to see and/or determine whether or not the expandable framework 12 is properly engaged with the projection 73 and/or the plurality of projections (e.g., ref. 73) of the stent holder 70.

Returning to FIG. 4, in some embodiments, the stent holder 70 may comprise a visual contrast region 86 (shown in FIG. 4 via dotted shading) proximate the projection 73. In some embodiments, the visual contrast region 86 may at least partially surround the projection 73 and/or the recess 74. In some embodiments, the visual contrast region 86 may extend more than halfway around the projection 73 and/or the recess 74. In some embodiments, the visual contrast region 86 may be spaced apart from the projection 73. In some embodiments, the visual contrast region 86 may generally appear as and/or resemble a U-shaped feature. In some embodiments, the visual contrast region 86 may generally appear as and/or resemble a squared-off U-shape. In some embodiments, the visual contrast region 86 may generally appear as and/or resemble a U-shape having a rounded (or partially rounded) base portion. Other configurations are also contemplated.

In at least some embodiments, the visual contrast region 86 may be visible without electronic imaging equipment (e.g., to the naked eye, with or without magnification). In some embodiments, the visual contrast region 86 may be disposed on an outer surface of the stent holder 70. In some embodiments, the visual contrast region 86 may be etched onto the outer surface of the stent holder 70. In some embodiments, the visual contrast region 86 may be mechanically etched onto the outer surface of the stent holder 70. In some embodiments, the visual contrast region 86 may be laser etched onto the outer surface of the stent holder 70. In some embodiments, the visual contrast region 86 may be chemically etched onto the outer surface of the stent holder 70.

As discussed herein, in some embodiments, the stent holder 70 may be formed from stainless steel. In some embodiments, the stent holder 70, the projection 73, and/or the recess 74 may have a shiny or polished finish or appearance (e.g., via laser polishing, plating, buffing, etc.). In some embodiments, the visual contrast region 86 may comprise a pigment. In some embodiments, the pigment may be a dark color, a light-absorbing pigment, etc. In some embodiments, the stainless steel may comprise a pigment. In some embodiments, etching the visual contrast region 86 may enhance and/or “amplify” the pigment of the stainless steel, thereby making the visual contrast regions appear even darker than etching alone. In some embodiments, a solution of 3% sodium borate or sodium bicarbonate may be added to the surface of the stent holder 70 in the visual contrast region 86 prior to etching. Sodium borate, when heated, creates sodium oxide and boron oxide which may react with carbon in the stainless steel of the stent holder 70, thereby reducing the burning point of the stainless steel and allowing for a darker etch with less time and lower laser power. Sodium, bicarbonate, when heated, breaks down to form sodium carbonate which reacts with natural pigments found in the stainless steel and darkens the natural pigments under laser processing. Other configurations are also contemplated. In some embodiments, the pigment may be added to the visual contrast region 86 after etching. In some embodiments, a black oxide may be added to the visual contrast region 86 after etching. In some embodiments, the pigment may comprise an enamel or an ink, which is and/or shall be biocompatible, applied to and/or buffed into the visual contrast region 86 after etching. In some embodiments, the visual contrast region 86 may comprise a matte finish. In some embodiments, the visual contrast region 86 may have a darker visual appearance than the projection 73 and/or an outward facing surface of the projection 73.

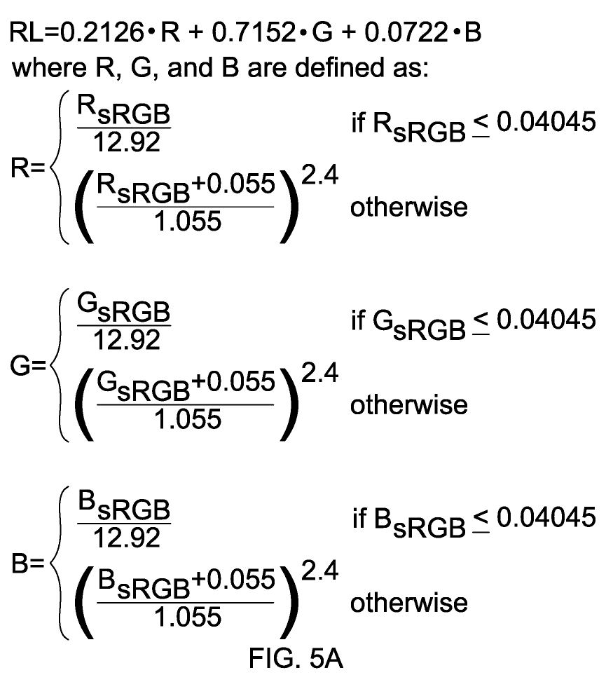

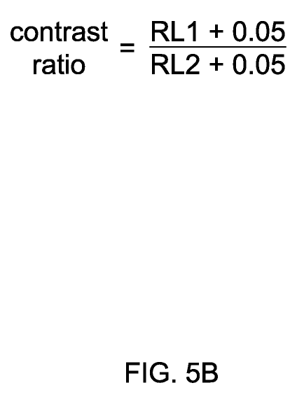

In some embodiments, a relative luminance of the outward facing surface of the projection 73 (or a non-etched surface of the stent holder 70) compared to and/or divided by a relative luminance of the visual contrast region 86 at least partially surrounding the projection 73 may define a contrast ratio of the outward facing surface of the projection 73 (or a non-etched surface of the stent holder 70) to the visual contrast region 86. The relative luminance (RL) may be calculated using red, green, and black values (e.g., RGB Value) obtained from areas being compared (e.g., the projection 73 (or a non-etched surface of the stent holder 70) and the visual contrast region 86). The RGB Value may be measured using software, for example. Examples of equations and/or calculations used to determine relative luminance and contrast ratio may be seen in FIGS. 5A-5B, wherein RL1 (e.g., FIG. 5B) is the relative luminance of the outward facing surface of the projection 73 (or a non-etched surface of the stent holder 70) and RL2 (e.g., FIG. 5B) is the relative luminance of the visual contrast region 86. Both relative luminance values (e.g., RL1 and RL2) are calculated using the equations and/or calculations shown in FIG. 5A.

In some embodiments, the contrast ratio of the outward facing surface of the projection 73 (or a non-etched surface of the stent holder 70) to the visual contrast region 86 may be at least 3:1 (e.g., the relative luminance of the outward facing surface of the projection 73 divided by the relative luminance of the visual contrast region 86 may be at least 3). In some embodiments, the contrast ratio of the outward facing surface of the projection 73 (or a non-etched surface of the stent holder 70) to the visual contrast region 86 may be at least 7:1 (e.g., the relative luminance of the outward facing surface of the projection 73 divided by the relative luminance of the visual contrast region 86 may be at least 7). In some embodiments, the contrast ratio of the outward facing surface of the projection 73 (or a non-etched surface of the stent holder 70) to the visual contrast region 86 may be at least 12:1 (e.g., the relative luminance of the outward facing surface of the projection 73 divided by the relative luminance of the visual contrast region 86 may be at least 12). Other configurations are also contemplated.

Additionally, in some embodiments, the contrast ratio and/or the relative luminance (RL) values may be calculated with respect to the replacement heart valve implant 10 compared to the visual contrast region 86. For example, the expandable framework 12 and the plurality of valve leaflets 20 and/or the inner skirt 22 and/or outer skirt 24 may at least partially obstruct the projection 73 when the replacement heart valve implant 10 and/or the expandable framework 12 is engaged with the stent holder 70 in the radially collapsed configuration. Proper positioning of the expandable framework 12 and/or the hook 15 around the projection 73 may be difficult to determine, particularly in a low-light environment. Accordingly, the contrast between the replacement heart valve implant 10 and the visual contrast region 86 may be useful for verifying the proper positioning of the replacement heart valve implant 10 and/or the expandable framework 12 with respect to the projection 73. The contrast ratio(s) discussed herein may also apply when the relative luminance (RL) of the replacement heart valve implant 10 is compared to the relative luminance (RL) of the visual contrast region 86.

As discussed herein, in some embodiments, the stent holder may comprise a plurality of projections (e.g., ref. 73) and a plurality of recesses (e.g., ref. 74) associated therewith. Accordingly, in some embodiments, the stent holder 70 may comprise visual contrast regions (e.g., ref. 86) proximate the plurality of projections (e.g., ref. 73). In some embodiments, the visual contrast regions (e.g., ref. 86) may at least partially surround the plurality of projections (e.g., ref. 73) and/or the plurality of recesses (e.g., ref. 74). In some embodiments, the visual contrast regions (e.g., ref. 86) may extend more than halfway around the plurality of projections (e.g., ref. 73) and/or the plurality of recesses (e.g., ref. 74). In some embodiments, the visual contrast regions (e.g., ref. 86) may be spaced apart from the plurality of projections (e.g., ref. 73).

In at least some embodiments, the visual contrast regions (e.g., ref. 86) may be visible without electronic imaging equipment (e.g., to the naked eye, with or without magnification). In some embodiments, the visual contrast regions (e.g., ref. 86) may be disposed on an outer surface of the stent holder 70. In some embodiments, the visual contrast regions (e.g., ref. 86) may be etched onto the outer surface of the stent holder 70. In some embodiments, the visual contrast regions (e.g., ref. 86) may be mechanically etched onto the outer surface of the stent holder 70. In some embodiments, the visual contrast regions (e.g., ref. 86) may be laser etched onto the outer surface of the stent holder 70. In some embodiments, the visual contrast regions (e.g., ref. 86) may be chemically etched onto the outer surface of the stent holder 70.

As discussed herein, in some embodiments, the stent holder 70 may be formed from stainless steel. In some embodiments, the stent holder 70, the plurality of projections (e.g., ref. 73), and/or the plurality of recesses (e.g., ref. 74) may have a shiny or polished finish or appearance (e.g., via laser polishing, plating, buffing, etc.). In some embodiments, the visual contrast regions (e.g., ref. 86) may comprise a pigment. In some embodiments, the pigment may be a dark color, a light-absorbing pigment, etc. In some embodiments, the stainless steel may comprise a pigment. In some embodiments, etching the visual contrast regions (e.g., ref. 86) may enhance and/or “amplify” the pigment of the stainless steel, thereby making the visual contrast regions appear even darker than etching alone. In some embodiments, a solution of 3% sodium borate or sodium bicarbonate may be added to the surface of the stent holder 70 in the visual contrast regions (e.g., ref. 86) prior to etching. Sodium borate, when heated, creates sodium oxide and boron oxide which may react with carbon in the stainless steel of the stent holder 70, thereby reducing the burning point of the stainless steel and allowing for a darker etch with less time and lower laser power. Sodium, bicarbonate, when heated, breaks down to form sodium carbonate which reacts with natural pigments found in the stainless steel and darkens the natural pigments under laser processing. Other configurations are also contemplated. In some embodiments, the pigment may be added to the visual contrast regions (e.g., ref. 86) after laser-etching. In some embodiments, a black oxide may be added to the visual contrast regions (e.g., ref. 86) after etching. In some embodiments, the pigment may comprise an enamel or an ink, which is and/or shall be biocompatible, applied to and/or buffed into the visual contrast regions (e.g., ref. 86) after etching. In some embodiments, the visual contrast regions (e.g., ref. 86) may comprise a matte finish. In some embodiments, the visual contrast regions (e.g., ref. 86) may have a darker visual appearance than the plurality of projections (e.g., ref. 73) and/or an outward facing surface of the plurality of projections (e.g., ref. 73).

In some embodiments, a relative luminance of the outward facing surface of the plurality of projections (e.g., ref. 73) (or a non-etched surface of the stent holder 70) compared to and/or divided by a relative luminance of the visual contrast regions (e.g., ref. 86) at least partially surrounding the plurality of projections (e.g., ref. 73) may define a contrast ratio of the outward facing surface of the plurality of projections (e.g., ref. 73) (or a non-etched surface of the stent holder 70) to the visual contrast regions (e.g., ref. 86). The relative luminance (RL) may be calculated using red, green, and black values (e.g., RGB Value) obtained from areas being compared (e.g., the plurality of projections (e.g., ref. 73) (or a non-etched surface of the stent holder 70) and the visual contrast regions (e.g., ref. 86)). The RGB Value may be measured using software, for example. Examples of equations and/or calculations used to determine relative luminance and contrast ratio may be seen in FIGS. 5A-5B, wherein RL1 (e.g., FIG. 5B) is the relative luminance of the outward facing surface of the plurality of projections (e.g., ref. 73) (or a non-etched surface of the stent holder 70) and RL2 (e.g., FIG. 5B) is the relative luminance of the visual contrast regions (e.g., ref. 86). Both relative luminance values (e.g., RL1 and RL2) are calculated using the equations and/or calculations shown in FIG. 5A.

In some embodiments, the contrast ratio of the outward facing surface of the plurality of projections (e.g., ref. 73) (or a non-etched surface of the stent holder 70) to the visual contrast regions (e.g., ref. 86) may be at least 3:1 (e.g., the relative luminance of the outward facing surface of the plurality of projections divided by the relative luminance of the visual contrast regions may be at least 3). In some embodiments, the contrast ratio of the outward facing surface of the plurality of projections (e.g., ref. 73) (or a non-etched surface of the stent holder 70) to the visual contrast regions (e.g., ref. 86) may be at least 7:1 (e.g., the relative luminance of the outward facing surface of the plurality of projections divided by the relative luminance of the visual contrast regions may be at least 7). In some embodiments, the contrast ratio of the outward facing surface of the plurality of projections (e.g., ref. 73) (or a non-etched surface of the stent holder 70) to the visual contrast regions (e.g., ref. 86) may be at least 12:1 (e.g., the relative luminance of the outward facing surface of the plurality of projections divided by the relative luminance of the visual contrast regions may be at least 12). Other configurations are also contemplated.

Additionally, in some embodiments, the contrast ratio and/or the relative luminance (RL) values may be calculated with respect to the replacement heart valve implant 10 compared to the visual contrast region 86. For example, the expandable framework 12 and the plurality of valve leaflets 20 and/or the inner skirt 22 and/or outer skirt 24 may at least partially obstruct the plurality of projections (e.g., ref. 73) when the replacement heart valve implant 10 and/or the expandable framework 12 is engaged with the stent holder 70 in the radially collapsed configuration. Proper positioning of the expandable framework 12 and/or the plurality of hooks (e.g., ref. 15) around the plurality of projections (e.g., ref. 73) may be difficult to determine, particularly in a low-light environment. Accordingly, the contrast between the replacement heart valve implant 10 and the visual contrast region 86 may be useful for verifying the proper positioning of the replacement heart valve implant 10 and/or the expandable framework 12 with respect to the plurality of projections (e.g., ref. 73). The contrast ratio(s) discussed herein may also apply when the relative luminance (RL) of the replacement heart valve implant 10 is compared to the relative luminance (RL) of the visual contrast region 86.

In addition, or alternatively, in some embodiments, the projection 73 may comprise a colored tip 77. In some embodiments, the colored tip 77 may comprise a paint disposed on an outward facing surface of the projection 73. In some embodiments, the paint may be a bright color, a high-visibility pigment, luminous and/or luminescent, etc. In some embodiments, the colored tip 77 may be configured to be visible without electronic imaging equipment when the distal portion of the expandable framework 12 of the replacement heart valve implant 10 is engaged with the projection 73 in the radially collapsed configuration and/or when the replacement heart valve implant 10 is constrained within the implant holding portion 60 of the delivery device 30. In some embodiments, the colored tip 77 may permit a user to more easily see and/or verify proper engagement of the distal portion of the expandable framework 12 of the replacement heart valve implant 10 with the projection 73 in the radially collapsed configuration and/or when the replacement heart valve implant 10 is constrained within the implant holding portion 60 of the delivery device 30.

In some embodiments, the plurality of projections (e.g., ref. 73) may each comprise a colored tip (e.g., ref. 77). In some embodiments, the colored tip (e.g., ref. 77) of each projection may comprise a paint disposed on an outward facing surface thereof. In some embodiments, the paint may be a bright color, a high-visibility pigment, luminous and/or luminescent, etc., and the paint is and/or shall be biocompatible. In some embodiments, the colored tip (e.g., ref. 77) of each projection may be configured to be visible without electronic imaging equipment when the distal portion(s) of the expandable framework 12 of the replacement heart valve implant 10 is engaged with the plurality of projections (e.g., ref. 73) in the radially collapsed configuration and/or when the replacement heart valve implant 10 is constrained within the implant holding portion 60 of the delivery device 30. In some embodiments, the colored tip (e.g., ref. 77) of each projection may permit a user to more easily see and/or verify proper engagement of the distal portion(s) of the expandable framework 12 of the replacement heart valve implant 10 with the plurality of projections (e.g., ref. 73) in the radially collapsed configuration and/or when the replacement heart valve implant 10 is constrained within the implant holding portion 60 of the delivery device 30.

Turning back again to FIGS. 2-3, in some embodiments, the handle assembly 40 comprises an actuation mechanism operatively engaged with the first rotatable knob 43. In some embodiments, the first rotatable knob 43 may be fixedly attached to the actuation mechanism. In some embodiments, the first rotatable knob 43 may be configured to rotate the actuation mechanism relative to the central longitudinal axis of the handle assembly 40. In some embodiments, rotation of the first rotatable knob 43 around the central longitudinal axis of the handle assembly 40 causes rotation of and/or rotates the actuation mechanism around the central longitudinal axis of the handle assembly 40.

In some embodiments, the first rotatable knob 43 may be configured to rotate the actuation mechanism relative to the central longitudinal axis of the handle assembly 40 to axially translate a first portion of the elongate shaft assembly 50 along the central longitudinal axis. In some embodiments, the first portion of the elongate shaft assembly 50 may comprise and/or may be the outer tubular member 52 and/or the proximal sheath 62. In some embodiments, rotation of the first rotatable knob 43 and/or the actuation mechanism may be configured to axially translate the first portion (e.g., the outer tubular member 52 and/or the proximal sheath 62) of the elongate shaft assembly 50 relative to the handle assembly 40. In some embodiments, rotation of the first rotatable knob 43 and/or the actuation mechanism in a first rotational direction may be configured to axially translate the first portion (e.g., the outer tubular member 52 and/or the proximal sheath 62) of the elongate shaft assembly 50 distally relative to the handle assembly 40 and/or toward the delivery configuration of the implant holding portion 60 (e.g., FIG. 2). In some embodiments, rotation of the first rotatable knob 43 and/or the actuation mechanism in a second rotational direction (e.g., opposite the first rotational direction) may be configured to axially translate the first portion (e.g., the outer tubular member 52 and/or the proximal sheath 62) of the elongate shaft assembly 50 proximally relative to the handle assembly 40 and/or toward the deployment configuration of the implant holding portion 60 (e.g., FIG. 3). In at least some embodiments, the first rotational direction may be clockwise as viewed from the proximal end 41 of the handle assembly 40 to the distal end 42 of the handle assembly 40, and the second rotational direction may be counterclockwise as viewed from the proximal end 41 of the handle assembly 40 toward the distal end 42 of the handle assembly 40.

In some embodiments, the handle assembly 40 may comprise a second actuation mechanism operatively engaged with the second rotatable knob 44. In some embodiments, the second rotatable knob 44 may be fixedly attached to the second actuation mechanism. In some embodiments, the second rotatable knob 44 may be configured to rotate the second actuation mechanism relative to the central longitudinal axis of the handle assembly 40. In some embodiments, rotation of the second rotatable knob 44 around the central longitudinal axis of the handle assembly 40 causes rotation of and/or rotates the second actuation mechanism around the central longitudinal axis of the handle assembly 40.

In some embodiments, the second rotatable knob 44 may be configured to rotate the second actuation mechanism relative to the central longitudinal axis of the handle assembly 40 to axially translate a second portion of the elongate shaft assembly 50 along the central longitudinal axis. In some embodiments, the second portion of the elongate shaft assembly 50 may comprise and/or may be the inner shaft 54 and/or the distal sheath 64. In some embodiments, rotation of the second rotatable knob 44 and/or the second actuation mechanism may be configured to axially translate the second portion (e.g., the inner shaft 54 and/or the distal sheath 64) of the elongate shaft assembly 50 relative to the handle assembly 40. In some embodiments, rotation of the second rotatable knob 44 and/or the second actuation mechanism in the first rotational direction may be configured to axially translate the second portion (e.g., the inner shaft 54 and/or the distal sheath 64) of the elongate shaft assembly 50 proximally relative to the handle assembly 40 and/or toward the delivery configuration of the implant holding portion 60 (e.g., FIG. 2). In some embodiments, rotation of the second rotatable knob 44 and/or the second actuation mechanism in the second rotational direction (e.g., opposite the first rotational direction) may be configured to axially translate the second portion (e.g., the inner shaft 54 and/or the distal sheath 64) of the elongate shaft assembly 50 distally relative to the handle assembly 40 and/or toward the deployment configuration of the implant holding portion 60 (e.g., FIG. 3). In some embodiments, axially translating the second portion (e.g., the inner shaft 54 and/or the distal sheath 64) of the elongate shaft assembly 50 in the distal direction to the deployment configuration may position a proximalmost end of the distal sheath 64 distal of the proximal head 72 of the stent holder 70.

In some embodiments, after shifting the implant holding portion 60 to the deployment configuration (e.g., FIG. 3) and/or after releasing the replacement heart valve implant 10, the implant holding portion 60 may be shifted from the deployment configuration (e.g., FIG. 3) of the implant holding portion 60 toward and/or to the delivery configuration (e.g., FIG. 2) of the implant holding portion 60 without the replacement heart valve implant 10 constrained therein to facilitate withdrawal of the delivery device 30 from the patient.

The materials that can be used for the various components of the delivery device and the various elements thereof disclosed herein may include those commonly associated with medical devices. For simplicity purposes, the following discussion refers to the system. However, this is not intended to limit the devices, components, and methods described herein, as the discussion may be applied to other elements, members, components, or devices disclosed herein, such as, but not limited to, the replacement heart valve implant, the delivery device, etc. and/or elements or components thereof.

In some embodiments, the system and/or components thereof may be made from a metal, metal alloy, polymer, a metal-polymer composite, ceramics, combinations thereof, and the like, or other suitable material.

Some examples of suitable polymers may include polytetrafluoroethylene (PTFE), ethylene tetrafluoroethylene (ETFE), fluorinated ethylene propylene (FEP), polyoxymethylene (POM; for example, DELRIN®), polyether block ester, polyurethane, polypropylene (PP), polyvinylchloride (PVC), polyether-ester (for example, ARNITEL®), ether or ester based copolymers (for example, butylene/poly(alkylene ether) phthalate and/or other polyester elastomers such as HYTREL®), polyamide (for example, DURETHAN® or CRISTAMID®), elastomeric polyamides, block polyamide/ethers, polyether block amide (PEBA; for example, PEBAX®), ethylene vinyl acetate copolymers (EVA), silicones, polyethylene (PE), MARLEX® high-density polyethylene, MARLEX® low-density polyethylene, linear low density polyethylene (for example, REXELL®), polyester, polybutylene terephthalate (PBT), polyethylene terephthalate (PET), polytrimethylene terephthalate, polyethylene naphthalate (PEN), polyetheretherketone (PEEK), polyimide (PI), polyetherimide (PEI), polyphenylene sulfide (PPS), polyphenylene oxide (PPO), poly paraphenylene terephthalamide (for example, KEVLAR®), polysulfone, nylon, nylon-12 (such as GRILAMID®), perfluoro(propyl vinyl ether) (PFA), ethylene vinyl alcohol, polyolefin, polystyrene, epoxy, polyvinylidene chloride (PVdC), poly(styrene-b-isobutylene-b-styrene) (for example, SIBS and/or SIBS 50A), polycarbonates, polyurethane silicone copolymers (for example, Elast-Eon® or ChronoSil®), biocompatible polymers, other suitable materials, or mixtures, combinations, copolymers thereof, polymer/metal composites, and the like. In some embodiments, the system and/or components thereof can be blended with a liquid crystal polymer (LCP).

Some examples of suitable metals and metal alloys include stainless steel, such as 304 and/or 316 stainless steel and/or variations thereof; mild steel; nickel-titanium alloy such as linear-elastic and/or super-elastic nitinol; other nickel alloys such as nickel-chromium-molybdenum alloys (e.g., UNS: N06625 such as INCONEL® 625, UNS: N06022 such as HASTELLOY® C-22®, UNS: N10276 such as HASTELLOY® C276®, other HASTELLOY® alloys, and the like), nickel-copper alloys (e.g., UNS: N04400 such as MONEL® 400, NICKELVAC® 400, NICORROS® 400, and the like), nickel-cobalt-chromium-molybdenum alloys (e.g., UNS: R30035 such as MP35-N® and the like), nickel-molybdenum alloys (e.g., UNS: N10665 such as HASTELLOY® ALLOY B2®), other nickel-chromium alloys, other nickel-molybdenum alloys, other nickel-cobalt alloys, other nickel-iron alloys, other nickel-copper alloys, other nickel-tungsten or tungsten alloys, and the like; cobalt-chromium alloys; cobalt-chromium-molybdenum alloys (e.g., UNS: R30003 such as ELGILOY®, PHYNOX®, and the like); platinum enriched stainless steel; titanium; platinum; palladium; gold; combinations thereof; or any other suitable material.

In at least some embodiments, portions or all of the system and/or components thereof may also be doped with, made of, or otherwise include a radiopaque material. Radiopaque materials are understood to be materials capable of producing a relatively dark image on a fluoroscopy screen or another imaging technique (e.g., ultrasound, etc.) during a medical procedure. This relatively dark image aids the user of the system in determining its location. Some examples of radiopaque materials can include, but are not limited to, gold, platinum, palladium, tantalum, tungsten alloy, polymer material loaded with a radiopaque filler, and the like. Additionally, other radiopaque marker bands and/or coils may also be incorporated into the design of the system to achieve the same result.

In some embodiments, a degree of Magnetic Resonance Imaging (MRI) compatibility is imparted into the system and/or other elements disclosed herein. For example, the system and/or components or portions thereof may be made of a material that does not substantially distort the image and create substantial artifacts (e.g., gaps in the image). Certain ferromagnetic materials, for example, may not be suitable because they may create artifacts in an MRI image. The system or portions thereof may also be made from a material that the MRI machine can image. Some materials that exhibit these characteristics include, for example, tungsten, cobalt-chromium-molybdenum alloys (e.g., UNS: R30003 such as ELGILOY®, PHYNOX®, and the like), nickel-cobalt-chromium-molybdenum alloys (e.g., UNS: R30035 such as MP35-N® and the like), nitinol, and the like, and others.

In some embodiments, the system and/or other elements disclosed herein may include a fabric material disposed over or within the structure. The fabric material may be composed of a biocompatible material, such a polymeric material or biomaterial, adapted to promote tissue ingrowth. In some embodiments, the fabric material may include a bioabsorbable material. Some examples of suitable fabric materials include, but are not limited to, polyethylene glycol (PEG), nylon, polytetrafluoroethylene (PTFE, ePTFE), a polyolefinic material such as a polyethylene, a polypropylene, polyester, polyurethane, and/or blends or combinations thereof.

In some embodiments, the system and/or other elements disclosed herein may include and/or be formed from a textile material. Some examples of suitable textile materials may include synthetic yarns that may be flat, shaped, twisted, textured, pre-shrunk or un-shrunk. Synthetic biocompatible yarns suitable for use in the present disclosure include, but are not limited to, polyesters, including polyethylene terephthalate (PET) polyesters, polypropylenes, polyethylenes, polyurethanes, polyolefins, polyvinyls, polymethylacetates, polyamides, naphthalene dicarboxylene derivatives, natural silk, and polytetrafluoroethylenes. Moreover, at least one of the synthetic yarns may be a metallic yarn or a glass or ceramic yarn or fiber. Useful metallic yarns include those yarns made from or containing stainless steel, platinum, gold, titanium, tantalum, or a Ni-Co-Cr-based alloy. The yarns may further include carbon, glass, or ceramic fibers. Desirably, the yarns are made from thermoplastic materials including, but not limited to, polyesters, polypropylenes, polyethylenes, polyurethanes, polynaphthalenes, polytetrafluoroethylenes, and the like. The yarns may be of the multifilament, monofilament, or spun types. The type and denier of the yarn chosen may be selected in a manner which forms a biocompatible and implantable prosthesis and, more particularly, a vascular structure having desirable properties.

In some embodiments, the system and/or other elements disclosed herein may include and/or be treated with a suitable therapeutic agent. Some examples of suitable therapeutic agents may include anti-thrombogenic agents (such as heparin, heparin derivatives, urokinase, and PPack (dextrophenylalanine proline arginine chloromethyl ketone)); anti-proliferative agents (such as enoxaparin, angiopeptin, monoclonal antibodies capable of blocking smooth muscle cell proliferation, hirudin, and acetylsalicylic acid); anti-inflammatory agents (such as dexamethasone, prednisolone, corticosterone, budesonide, estrogen, sulfasalazine, and mesalamine); antineoplastic/antiproliferative/anti-mitotic agents (such as paclitaxel, 5-fluorouracil, cisplatin, vinblastine, vincristine, epothilones, endostatin, angiostatin and thymidine kinase inhibitors); anesthetic agents (such as lidocaine, bupivacaine, and ropivacaine); anti-coagulants (such as D-Phe-Pro-Arg chloromethyl ketone, an RGD peptide-containing compound, heparin, anti-thrombin compounds, platelet receptor antagonists, anti-thrombin antibodies, anti-platelet receptor antibodies, aspirin, prostaglandin inhibitors, platelet inhibitors, and tick antiplatelet peptides); vascular cell growth promoters (such as growth factor inhibitors, growth factor receptor antagonists, transcriptional activators, and translational promoters); vascular cell growth inhibitors (such as growth factor inhibitors, growth factor receptor antagonists, transcriptional repressors, translational repressors, replication inhibitors, inhibitory antibodies, antibodies directed against growth factors, bifunctional molecules consisting of a growth factor and a cytotoxin, bifunctional molecules consisting of an antibody and a cytotoxin); immunosuppressants (such as the “olimus” family of drugs, rapamycin analogues, macrolide antibiotics, biolimus, everolimus, zotarolimus, temsirolimus, picrolimus, novolimus, myolimus, tacrolimus, sirolimus, pimecrolimus, etc.); cholesterol-lowering agents; vasodilating agents; and agents which interfere with endogenous vasoactive mechanisms.

It should be understood that this disclosure is, in many respects, only illustrative. Changes may be made in details, particularly in matters of shape, size, and arrangement of steps without exceeding the scope of the disclosure. This may include, to the extent that it is appropriate, the use of any of the features of one example embodiment being used in other embodiments. The scope of the disclosure is, of course, defined in the language in which the appended claims are expressed.

What is claimed is:

1. A delivery device for a replacement heart valve implant, comprising:
an elongate shaft assembly including an implant holding portion;
wherein the implant holding portion is configured to constrain a replacement heart valve implant in a radially collapsed configuration;
wherein the elongate shaft assembly comprises a stent holder including a projection configured to engage a distal portion of an expandable framework of the replacement heart valve implant when the replacement heart valve implant is constrained within the implant holding portion of the implant delivery system;
wherein the stent holder comprises a visual contrast region proximate the projection.

2. The delivery device of claim 1, wherein the visual contrast region is visible without electronic imaging equipment.

3. The delivery device of claim 1, wherein the visual contrast region is disposed on an outer surface of the stent holder.

4. The delivery device of claim 3, wherein the visual contrast region is etched onto the outer surface of the stent holder.

5. The delivery device of claim 1, wherein the visual contrast region at least partially surrounds the projection.

6. The delivery device of claim 5, wherein the visual contrast region extends more than halfway around the projection.

7. The delivery device of claim 1, wherein a contrast ratio of the visual contrast region to an outward facing surface of the projection is at least 3:1.

8. The delivery device of claim 7, wherein the contrast ratio of the visual contrast region to the outward facing surface of the projection is at least 7:1.

9. The delivery device of claim 7, wherein the contrast ratio of the visual contrast region to the outward facing surface of the projection is at least 12:1.

10. The delivery device of claim 1, wherein the visual contrast region has a darker visual appearance than the projection.

11. The delivery device of claim 1, wherein the stent holder is formed from a metallic material.

12. The delivery device of claim 11, wherein the metallic material comprises a pigment.

13. The delivery device of claim 11, wherein the visual contrast region comprises a pigment.

14. The delivery device of claim 1, wherein the visual contrast region comprises a matte finish.

15. A delivery device for a replacement heart valve implant, comprising:
an elongate shaft assembly including an implant holding portion;
wherein the implant holding portion is configured to constrain a replacement heart valve implant in a radially collapsed configuration;
wherein the elongate shaft assembly comprises a stent holder including a projection configured to engage a distal portion of an expandable framework of the replacement heart valve implant when the replacement heart valve implant is constrained within the implant holding portion of the implant delivery system;
wherein the projection comprises a colored tip.

16. The delivery device of claim 15, wherein the colored tip comprises a paint disposed on the projection.

17. The delivery device of claim 15, wherein the colored tip is configured to be visible when the distal portion of the expandable framework of the replacement heart valve implant is engaged with the projection.

18. A delivery device for a replacement heart valve implant, comprising:
an elongate shaft assembly including an implant holding portion;
wherein the implant holding portion is configured to constrain a replacement heart valve implant in a radially collapsed configuration;
wherein the elongate shaft assembly comprises a stent holder including a plurality of projections configured to engage a distal portion of an expandable framework of the replacement heart valve implant when the replacement heart valve implant is constrained within the implant holding portion of the implant delivery system;
wherein the stent holder comprises visual contrast regions at least partially surrounding each projection of the plurality of projections.

19. The delivery device of claim 18, wherein the visual contrast regions comprise etching on an outer surface of the stent holder.

20. The delivery device of claim 18, wherein a relative luminance of the plurality of projections divided by a relative luminance of the visual contrast regions at least partially surrounding each projection is at least 3.
